(19) United States
(12) Patent Application Publication
(10) Pub. No.: US 2014/0046162 A1
(43) Pub. Date: Feb. 13, 2014

(54) System and Method for Measuring Absolute Cardiac Volume Using a Combined Blood and Muscle Conductivity Model

(71) Applicants: John Rene Dubois, London (CA); Anil Tharian George Kottam, London (CA); Philip Chadwick Hodgson, London (CA); Peter David Plouf, London (CA); Geoffrey Kim Wood, London (CA)

(72) Inventors: John Rene Dubois, London (CA); Anil Tharian George Kottam, London (CA); Philip Chadwick Hodgson, London (CA); Peter David Plouf, London (CA); Geoffrey Kim Wood, London (CA)

(73) Assignee: Transonic Scisense Inc., London (CA)

(21) Appl. No.: 14/114,457

(22) PCT Filed: Apr. 30, 2012
(86) PCT No.: PCT/CA12/50270

§ 371 Date: Oct. 28, 2013

Related U.S. Application Data
(60) Provisional application No. 61/480,828, filed on Apr. 29, 2011.

(51) Int. Cl. A61B 5/0295; A61B 5/042; A61B 5/00

(52) U.S. Cl. CPC A61B 5/0295; A61B 5/6852; A61B 5/686; A61B 5/0004; A61B 5/0422
USPC 600/374; 600/508

(57) ABSTRACT

A method for real-time observation of absolute ventricular volume wherein electrical measurements are made with a tetra polar catheter and compared to electrical measurements in a database, said database being assembled using electric field theory to predict the non-linear relation between blood volume and electrical measurements between a fist limit characterized by an infinitely thick volume of blood and second limit characterized by infinitely thick tissue completely surrounding the catheter. The calculations are performed under the assumption that the blood is surrounded by an infinitely thick region of tissue.

This application claims priority from U.S. Provisional Application No. 61/480,828 filed on Apr. 29, 2011, the contents of which are incorporated herein by reference.

### TECHNICAL FIELD

The following relates generally to systems and methods for measuring absolute cardiac volume.

### BACKGROUND

Cardiac researchers often need to know the exact volume of blood throughout the cardiac cycle. This can be accomplished using a technique known as conductance catheter based volumetry. All matter has a characteristic conductivity that specifies the ability of the matter to conduct a current. Conductance volumetry and admittance volumetry are techniques that can be used to measure real-time changes in cardiac chamber volume in a live, beating heart based on the current flow through the blood and tissues in a cardiac chamber. A tetrapolar catheter incorporating a pair of excitation electrodes and recording electrodes is typically used for conductance volumetry of a heart ventricle. The signals received by the recording electrodes are correlated to volume through the use of Baan's Volume Equation. The volume measurements can be used to determine the stroke volume, which is the volume of blood that is pumped for each cardiac cycle or each “beat” of the heart. Stroke volume is an important indicator of various cardiac conditions, as it correlates with the cardiac function.

Due to the significant size of the tetrapolar catheter, its utility has typically been restricted to mammals of larger size, including humans. More recently however, tetrapolar catheters have been miniaturized to a point where they may be applied to small rodents, a typical subject of cardiovascular research. With these catheters, the recording electrodes may be spaced as little as a few millimetres apart and span the long axis of the ventricle.

Due to the proximity of the electrodes to adjacent heart tissue, the electric field is not confined to the space occupied by blood. The measured conductance or admittance is often a combination of the blood and muscle values. This results in an unpredictable overestimation of the cardiac volume when correlated to a model. The difficulty in separating the tissue component of the conductance or admittance from the blood component lies in the fact that the heart's tissues move throughout the heartbeat. The relationship between the measured conductance and the blood volume is non-linear. Because existing technology relies on a linear model of the relationship between conductance and volume, errors are particularly great close to the limits, where in an infinite volume of blood, the conductance is predicted to be infinite. Current leakage into the myocardium tissue may therefore result in an overestimation of volume.

Existing methods of more accurately estimating this volume rely on separating the blood contribution from the tissue contribution using the phase signal delay between the transmitted current signal and the observed admittance signal. In this approach, the time delay is attributed solely to the muscle component and the calibration is performed based on scheme developed by measuring electrical muscle properties. Alternatively, multi-frequency observations of admittance that attributes signal attenuation to muscle incursion into the electric field can be made. However, both of these methods suffer from the difficulties in measuring the electrical muscle properties of the heart. In particular, measuring electrical muscle properties of smaller animals, such as mice, is especially difficult.

Another method is the injection of a saline bolus to alter blood conductivity and extrapolate a line of identity. This approach is problematic due to its reliance on a linear model. Further, this method doesn't take into account considerable human error and the variation of the electric field throughout the heartbeat.

It is therefore an object of the present invention to obviate or mitigate the above disadvantages.

### SUMMARY

In one aspect, there is provided a method for determining absolute volume of a physical lumen, the method comprising: obtaining a plurality of admittance measurements; determining a first admittance value representative of the lumen at a peak volume; determining a second admittance value representative of the lumen at a lowest volume; and determining an instantaneous volume of the lumen using the first and second admittance values and at least one predetermined calibration parameter.

In another aspect, there is provided a computer readable medium comprising computer executable instructions for performing the method.

In yet another aspect, there is provided a system comprising a processor and a memory, the memory comprising computer executable instructions for performing the method.

### BRIEF DESCRIPTION OF THE DRAWINGS

Embodiments will now be described by way of example only with reference to the appended drawings wherein:

FIG. 1 is a diagram of a tetrapolar catheter attached to a control module.

FIG. 2 is a diagram of a tetrapolar catheter in a heart.

FIG. 3 is an enlarged view of the tetrapolar catheter.

FIG. 4 is a view of the tetrapolar catheter showing the electric field when completely surrounded by tissue.

FIG. 5 is a view of the tetrapolar catheter showing the electric field when completely surrounded by blood.

FIG. 6 is a view of the tetrapolar catheter showing the electric field permeating a cylinder of blood and into the tissue of infinite thickness.

FIG. 7 is a chart showing the electrical admittance measured by the electrodes of the tetrapolar catheter versus the radius of the blood cylinder.

FIG. 8 is a chart showing the electrical admittance values correlated to the heartbeat to determine the end diastolic and end systolic values of admittance.

FIG. 9 is a chart similar to that of FIG. 6 showing points of the heart cycle corresponding to the end diastole and end systole.

FIG. 10 is a diagram illustrating the system used to calculate volume using an implanted catheter

FIG. 11 is a diagram similar to FIG. 10 illustrating the system used to calculate volume using an implanted catheter and implanted control module.

FIG. 12 is a diagram similar to FIG. 10 illustrating the system used to calculate volume using an implanted catheter, implanted control module and implanted blood pressure monitor.

FIG. 13 is a diagram similar to FIG. 12 illustrating the system used to calculate volume wherein the volume processing module is fixed to the subject.

FIG. 14 is a diagram similar to FIG. 13 illustrating the system used to calculate volume wherein the volume processing module is implanted in the subject.

FIG. 15 is a diagram similar to FIG. 14 illustrating the system used to calculate volume wherein the volume calculation is delivered to an artificial organ.

### DETAILED DESCRIPTION

It will be appreciated that for simplicity and clarity of illustration, where considered appropriate, reference numerals may be repeated among the figures to indicate corresponding or analogous elements. In addition, numerous specific details are set forth in order to provide a thorough understanding of the example embodiments described herein. However, it will be understood by those of ordinary skill in the art that the example embodiments described herein may be practised without these specific details. In other instances, well known methods procedures and components have not been described in detail so as not to obscure the example embodiments described herein. Also, the description is not to be considered as limiting the scope of the example embodiments described herein.

In the following, methods and systems are provided to measure absolute ventricular volume in real time over the course of the cardiac cycle. It will be appreciated that although the following examples are provided in the context of a catheter, the principles discussed herein are equally applicable to other devices that emit and measure electrical signals. For example, the device could comprise more than four electrodes, wherein the electrodes in use are selectable based on the geometry of the lumen being studied. The means to measure and emit electrical signals may also, for example, comprise a catheter having four or more electrodes that have been individually inserted in the lumen and spaced at a known distance.

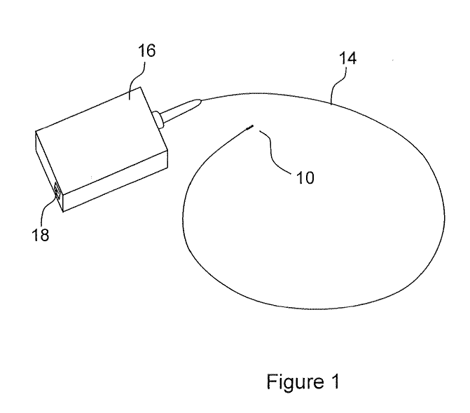

Referring therefore to FIG. 1, a tetrapolar catheter [10] is shown attached to a control module [16] via connecting line [14]. A port [18] allows information to be transferred to and from the controller module and can recharge a battery if the control module [16] comprises a battery. The length of connecting line [14] can vary depending on its use. The diameters of connecting line [14] and catheter [10] vary depending on the subject and the quantity of fluid that is to be delivered.

Although the examples herein utilize a tetrapolar catheter, it can be appreciated that other types of catheters may be used.

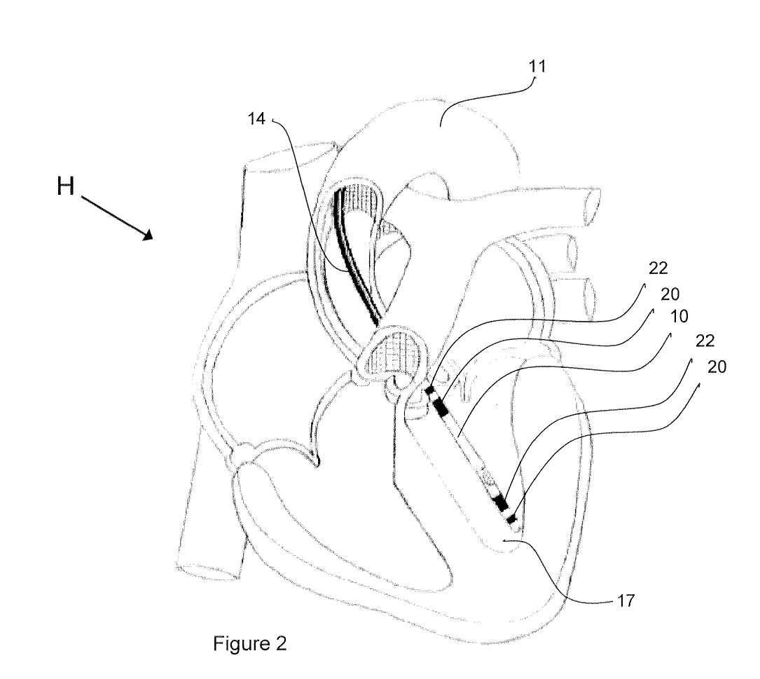

Referring to FIG. 2, the catheter [10] may comprise more than four electrodes in order to vary the effective length of the catheter. For example, the catheter may have 6 electrodes, where in the left ventricle [17], the distal excitation electrode [20] and sensing electrode [22] are used with whichever excitation electrode [20] is in the ventricle [17] but closest to the aortic valve and its adjacent sensing electrode [22]. Irrespective of the size of the left ventricle [17], the distal excitation electrode [20] and its adjacent sensing electrode [22] are used along with either the excitation electrode [20] closest to the connecting line [14] and its adjacent sensing electrode [22] or the middle excitation electrode [20] and its adjacent sensing electrode [22]. In larger left ventricles [17], the excitation electrode [20] closest to the connecting line [14] and its adjacent sensing electrode [22] are used whereas in smaller ventricles [17], the middle excitation electrode [20] and its adjacent sensing electrode [22] are used.

The tetrapolar catheter [10] of FIG. 1 is shown inserted into the left cardiac ventricle [17]. The catheter [10] is inserted through the aorta [11] through means well known in the art and spans the long axis of the ventricle [17]. The catheter [10] may also be inserted through the myocardium to be located within the ventricle [17]. Note however that though examples shown herein are in reference to the catheter [10] being placed in the left ventricle [17], it will be understood to a person skilled in the art that the catheter [10] is not limited to placement within the left ventricle [17]. The same concepts could be applied to associate the admittance of the tetrapolar catheter [10] in any of the other three chambers of the heart with the cardiac cycle relative to the chamber in which it is installed. These same concepts could also be applied to any physical lumen where tissue incursion would distort the volume calculation of a catheter [10]. For example, other physical lumen where tissue incursion would distort the volume calculation of a catheter [10] includes a blood vessel and the bladder.

The catheter [10] comprises a first excitation electrode [20] which is placed on the upper portion of the left ventricle [17] proximal to the connecting line [14] and a second excitation electrode [20] placed at the lower portion of the left ventricle [17], at the distal end of the catheter [10]. A first sensing electrode [22] is disposed adjacent to the first excitation electrode [20] and a second sensing electrode is disposed adjacent to the second excitation electrode [20] and both sensing electrodes are located between the pair of excitation electrodes [20].

Due to the placement of the electrodes, the electric field formed by the excitation electrodes [20] extends across the entire left ventricle [17]. As the left ventricle [17] is composed of myocardium tissue [15], which has a different characteristic conductivity than the blood located within the ventricle [17], the electric field sensed at the sensing electrodes [22] will differ depending on the contribution from the myocardium tissue component of the conductance and the blood component of the conductance.

Oxygenated blood from the lungs enters the left atrium [19], where it is delivered through the bicuspid valve [21] into the left ventricle [17]. When the left atrium [19] has dispelled its load of blood, the left ventricle [17] contains its maximal volume of blood. At this stage, the volume of blood in the left ventricle [17] is at its maximum, which is known as the end diastolic volume (EDV). The left ventricle [17] then contracts to force the oxygenated blood through the aorta [11] to be distributed throughout the body. Blood is prevented from flowing from the left ventricle [17] to the left atrium [19] by the bicuspid valve [21]. At the point of the cardiac cycle where the left ventricle [17] has contracted to its smallest volume, it contains only the end-systolic volume (ESV) of blood. The total volume of blood that is pumped for each contraction of the left ventricle [17] is known as the stroke volume (SV). The SV is equivalent to the difference between the end-diastolic volume and end-systolic volume of blood in the left ventricle [17].

As the volume of blood within the left ventricle [17] varies throughout the cardiac cycle, so too does the myocardial contribution to the conductance and the blood contribution to the conductance. At the EDV, the blood contribution to the conductance will be at its greatest and at the ESV, the myocardial contribution to the conductance is at its greatest. For example, in an adult human heart, the EDV value could be, for example, 120 mL and the ESV could be, for example, 50 mL. In this case, the ventricular SV would be 70 mL. Given this, it is particularly important to consider the varying contributions of blood and myocardium in determining the stroke volume of the left ventricle [17].

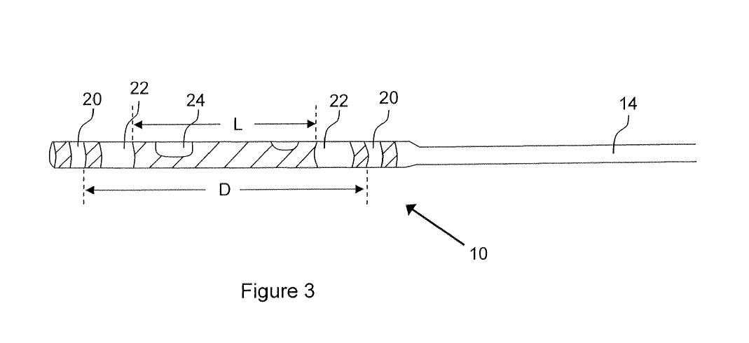

An enlarged view of the tetrapolar catheter [10] in the left ventricle [17] of FIG. 2 is shown in FIG. 3, a first excitation electrode (20) is located circumferentially about the distal end of the catheter [10] and a second excitation electrode is located at the proximal end of the catheter [10] in the ventricle [17]. The excitation electrodes [20], separated by distance D, provide an alternating current (AC) signal, which is detected at the voltage sensing electrodes [22]. The voltage sensing electrodes [22] are separated by distance L. As stated above, the catheter [10] is not limited to being tetrapolar and may comprise, for example, 6 or 14 electrodes. By selecting which of the catheter's [10] electrodes are used, the effective D and L values can be varied to suit the geometry of the ventricle [17]

This AC signal provided by the excitation electrodes [22] is not restricted to a given frequency. For example, the AC signal could be varied over the course of measurements from, 2 kHz to 30 kHz. The AC signal could also comprise signal components at several frequencies, for example, components from 2 kHz to 30 kHz at 7 kHz intervals. The sensing electrodes [22] are placed adjacent to, and between, the excitation electrodes [20]. When placed in the heart, the voltage sensed at the sensing electrodes [22] is dependent on the amount of blood surrounding the catheter [10].

The body of the left ventricle [17] can be approximated as a cylinder. Using this approximation, the catheter [10] can be modeled in the left ventricle [17] as a system of concentric cylinders where the inner cylinder represents blood and is characterized by radius r and the outer cylinder represents the myocardial tissue of the heart. In this model, the admittance or conductance of the surrounding medium has a contribution from the blood cylinder and a contribution from the myocardium. The ratio of these contributions is proportional to the ratio of the thickness of each medium. To accurately measure the volume of blood in the left ventricle [17], the volume of the inner cylinder must be determined so as to not include contributions from the myocardium in the result for the volume calculations.

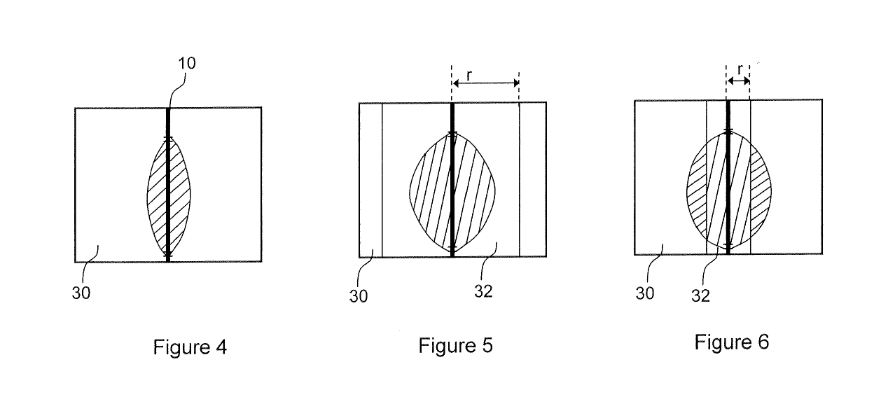

Turning to FIG. 4, a ventricle [17] that is completely empty of blood is shown, and hence, the entire admittance will be derived from the tissue calculation. Since, in this model, the ventricle [17] is completely devoid of blood, the admittance depends only on the myocardial contribution. The myocardial admittance in the absence of blood contribution is represented by Yₐ₀, which can be expressed using the following equation:

$${Y}_{ao} = - \frac{π d ( {L}^{2} - {D}^{2} ) ( {σ}_{m} + ω {ɛ}_{m} i )}{2 L \sqrt{4 {a}_{o}^{2} + {D}^{2}}}$$

Where D and L represent the spacing between the field generating electrodes [20] and the spacing between the field sensing electrodes [22] respectively, as illustrated in FIG. 3; aₒ represents the radius of the catheter [10], σₘ represents the conductivity of muscle, εₘ represents the permittivity of muscle, and ω represents the frequency of the applied field.

FIG. 5 shows the concentric cylinder model of the ventricle [17] when it is filled with blood to a point where the electric field exists entirely within the blood cylinder and hence, the myocardial contribution to the admittance measurements is zero. Since the electric field permeates only the blood, the admittance measured depends only on the blood contribution and not the myocardial contribution. Given that the electric field exists entirely within the inner concentric cylinder that corresponds to blood, this essentially represents a catheter [10] in an infinite volume of blood, and hence, the admittance is represented by Y_{inf-}Y_{inf} can be calculated using the following equation:

$${Y}_{inf} = - \frac{π d ( {L}^{2} - {D}^{2} ) ( {σ}_{b} + ω {ɛ}_{b} i )}{2 L \sqrt{4 {a}_{o}^{2} + {D}^{2}}}$$

Where σ_{b} represents the conductivity of blood, and ε_{b} represents the permittivity of blood.

Empirically, Y_{inf} can be determined for a catheter [10] of given dimensions by immersing the catheter [10] in a large volume of fluid with known electrical properties. For example, the catheter [10] could be immersed in a large volume of blood or in a large volume of liquid that has similar electrical properties to blood. Yₐₒ in contrast, is impractical to determine directly in an empirical way, as this is a measure of the admittance in a ventricle [17] that is devoid of blood. However, Yₐₒ can be written in terms of Y_{inf} by manipulating the equations for these admittance limits. Specifically,

$${Y}_{ao} = {Y}_{inf} \frac{( {σ}_{m} + ω {ɛ}_{m} i )}{( {σ}_{b} + ω {ɛ}_{b} i )}$$

The conductivity and permittivity of myocardium can be readily determined experimentally. Hence, from knowing Y_{inf} and measured values for the conductivity and permittivity of blood and myocardium, Yₐₒ can be determined.

This method and system described herein provides an improvement over prior methods in that it does not predict a linear relationship between the admittance of the catheter [10] and the diameter of the inner cylinder, which represents the blood. For example, using the classical Baan's approach to conductance volumetry would yield an infinite admittance when the catheter [10] is placed in an infinitely thick cylinder of blood, a result that is demonstrably false.

Referring to FIG. 6, which illustrates a concentric cylindrical model of a ventricle [17] whose admittance is dependent on a blood contribution and a myocardial contribution, as the electric field extends beyond the boundary of the blood cylinder. The radius r corresponds to the radius of the inner concentric cylinder which corresponds to the radius of blood surrounding the catheter [10]. The radius of the inner cylinder, r, varies depending on the current phase of the cardiac cycle, as r is dependent on the volume of blood in the ventricle [17]. The admittance measured in a model that depends on the blood contribution as well as the myocardial contribution will vary with time over the cardiac cycle as r expands and contracts. Since the ventricle [17] contains at least the ESV, it is never completely void of blood, and hence r is always greater than zero. The value of the measured admittance is characterized by Y(t), as the admittance is a function of time depending on the phase of the cardiac cycle.

The combined admittance as a function of time measured using the catheter [10] can be expressed as a sum of the blood contribution to the admittance and the myocardial contribution to the admittance as follows:

$$Y ( t ) = \frac{π d ( {D}^{2} - {L}^{2} )}{4 L} [ ( {σ}_{b} + i ω {ɛ}_{b} ) ( \frac{1}{\sqrt{{a}_{o}^{2} \frac{{D}^{2}}{4}}} - \frac{1}{\sqrt{{R}^{2} + \frac{{D}^{2}}{4}}} ) + ( {σ}_{m} + i ω {ɛ}_{m} ) ( \frac{1}{\sqrt{{R}^{2} + \frac{{D}^{2}}{4}}} ) ]$$

Where D and L represent the spacing between the field generating electrodes [20] and the spacing between the field sensing electrodes [22] respectively, as illustrated in FIG. 3, aₒ represents the radius of the catheter [10], σ_{b}, σₘ represent the conductivity of blood and muscle respectively, ε_{b} and εₘ represent the permittivity of blood and muscle respectively, ω represents the frequency of the applied field and R represents the radius of the cylinder of blood.

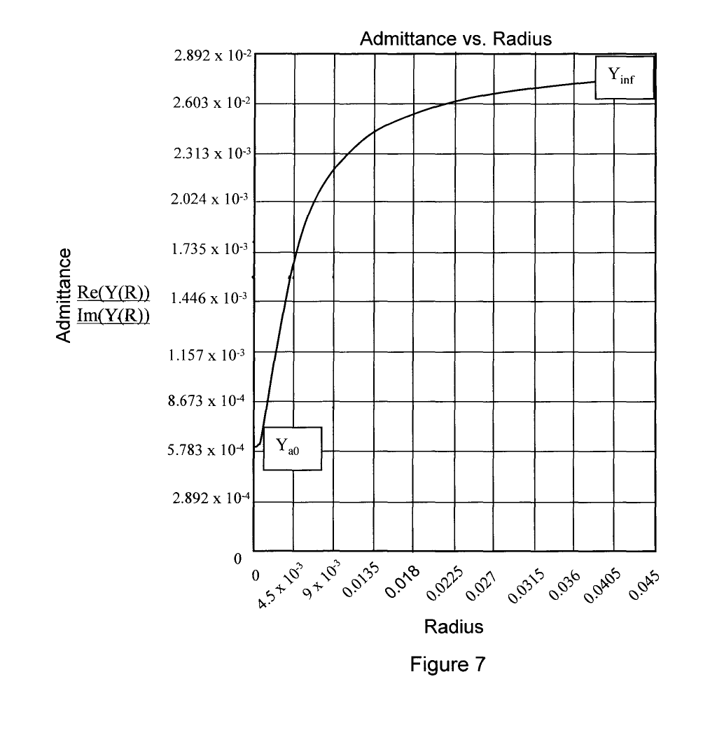

FIG. 7 shows the admittance measured by the sensing electrodes [22] of a catheter [10], separated into its real and imaginary components, as a function of the radius of the cylinder of blood. This plot is valid for a given catheter [10] and electrode [20, 22] geometry, as observed with a tetrapolar admittance catheter [10] and an admittance meter. This plot represents the changing value of admittance Y(t) as the thickness of the blood cylinder transitions from 0, corresponding to the Yₐ₀ admittance, to an infinity, which corresponds to the asymptotically limited Y_{inf} admittance value. All regions of the curve between these two limits represent an admittance that has a blood component and a myocardial component.

It may be noted that the real component of the admittance versus radius curve (Y(t)) is monotonically increasing between the admittance value of Yₐ₀ and the admittance value of Y_{inf} whereas the imaginary component of the admittance versus radius curve is monotonically decreasing. Since both functions are monotonic, at any radius of the cylinder of blood, there is a single corresponding admittance value. Hence, given accurate calibration factors, the measured admittance can be mapped to determine an absolute volume of blood.

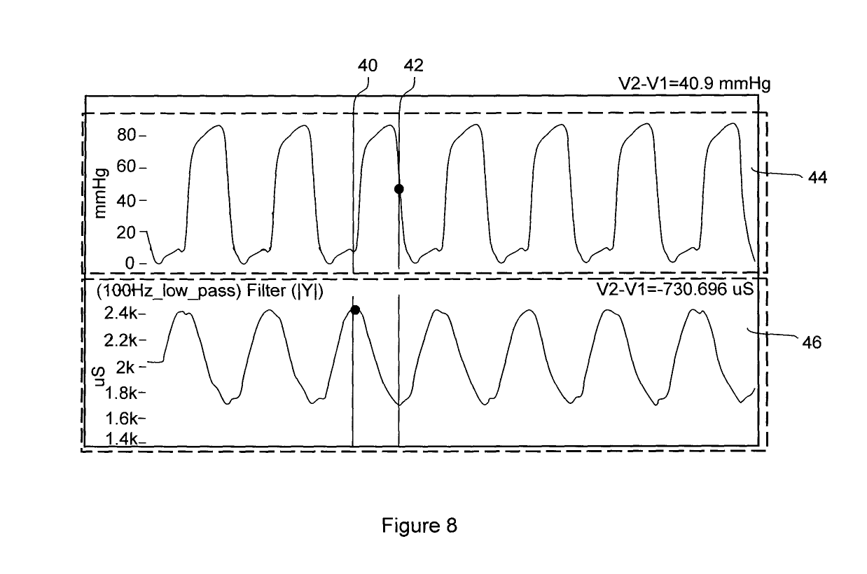

To calculate the blood volume, it is necessary to know the SV, which is a commonly measured value and can be determined via echocardiography, flow meter or other technologies known in the art. Turning to FIG. 8, the admittance relative to the cardiac cycle can be determined by measuring blood pressure [44] and admittance [46] simultaneously. The blood pressure tracing is used to accurately locate the ESV and EDV points on the admittance trace. The admittance values [46] shown on the plot of FIG. 8 correspond to the admittance values of FIG. 7 but, as the measurements are performed on a living heart, the admittance values are held between the values associated with the end of ventricular systole and the end of ventricular diastole.

Through a comparison of the time-dependent admittance data measured from the catheter [10] to the cardiac cycle, the admittance values and the constituent blood and myocardial contributions at specific phases of the cardiac cycle can be determined. In particular, the end systole and end diastole admittance values are determined by locating these points on the blood pressure versus time plot [44]. The leftmost cursor line [40] represents the end of ventricular diastole and the rightmost cursor line [42] represents the end of ventricular systole, both of which can be determined from the blood pressure versus time plot [44]. By comparing the pressure and the admittance over the same temporal frame [44, 46], the measured admittance, which comprises blood and myocardial components, can be determined at these two critical points in the cardiac cycle.

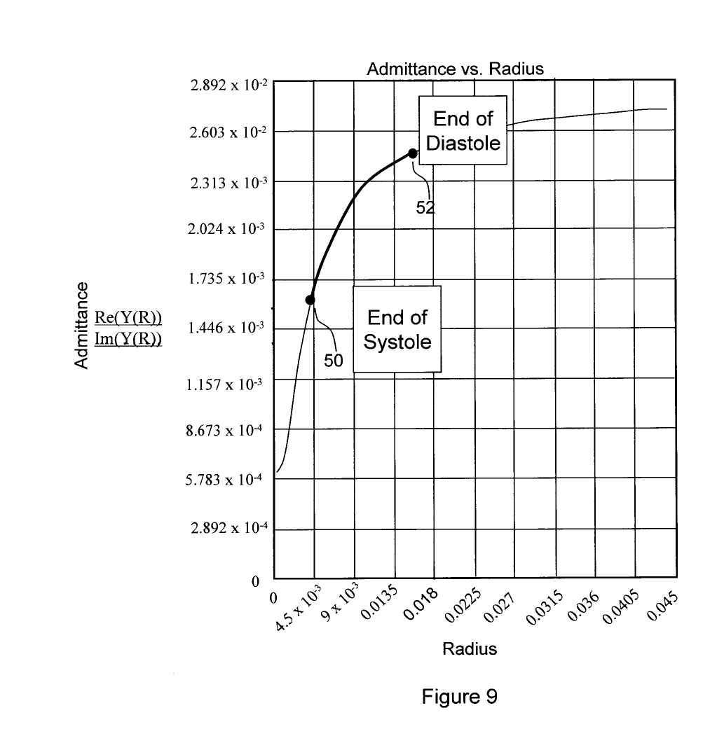

Referring now to FIG. 9, which is derived from the plot of FIG. 7, points on the curve corresponding to the end of ventricular diastole [52] and the end of ventricular systole [50] have been identified based on the correlation between the blood pressure tracing to the measured admittance tracing, as explained above and shown in FIG. 8. These two points correspond to the limits of the admittance curve in the left ventricle [17] of a living, beating heart, as the admittance at the end of ventricular systole [50] is based on a particular radius of the blood cylinder with a volume equal to the ESV and similarly, the end of ventricular diastole [52] is based on a specific radius of the blood cylinder with a volume equal to the EDV. Hence, since the volume of blood in the left ventricle [17] varies between the ESV and the EDV, so too does the admittance vary as a function of the volume of blood in the left ventricle [17], between the admittance at the end of ventricular systole and the admittance at the end of ventricular diastole.

To determine the instantaneous volume of blood in the ventricle [17], the measured admittance is used. The stroke volume is an empirical normalization factor that ensures that the difference in volumes corresponding to the admittance at the end of ventricular systole and the admittance at the end of ventricular diastole is equal to the measured stroke volume.

The actual volume of blood in the left ventricle [17] can then be calculated from the admittance data of FIG. 8 using the admittance measured at the end of ventricular systole and the end of ventricular diastole according to the following:

$$Vol = π {La}_{o}^{2} + \frac{SV ( {Y}_{meas .} - {Y}_{ao} ) {( {Y}_{ED} - {Y}_{inf} )}^{2} {( {Y}_{ES} - {Y}_{inf} )}^{2} ( {Y}_{meas .} + {Y}_{ao} - 2 {Y}_{inf} )}{{( {Y}_{meas .} - {Y}_{inf} )}^{2} ( {Y}_{ED} - {Y}_{ES} ) {( {Y}_{ao} - {Y}_{inf} )}^{2} ( {Y}_{ED} + {Y}_{ES} - 2 {Y}_{inf} )}$$

Where Yₘₑₐₛ, is the instantaneous value of admittance as measured with the catheter [10], Y_{ED} is the admittance value at the end of ventricular diastole, determined from correlating the admittance value to the blood pressure trace over the cardiac cycle and Y_{ES} is the admittance value at the end of ventricular systole, determined from correlating the admittance value to the blood pressure trace over the cardiac cycle.

Several advantages are inherent in relating the measured admittance values to the volume using the above equation. First, since all measurements for the calibration are taken with the same physical catheter [10] and associated instrumentation, any calibration offsets are removed due to the subtraction of the admittance values from every term. Second, because any scaling factor would be present in both the numerator and the denominator of the aforementioned equation, scaling factors associated with the catheter [10] would cancel and not affect the volume calculation. Third, no phase signal is required to produce absolute volume numbers. Since no phase signal is required, apart from its conductivity and permittivity, there is no need to measure complex electrical muscle properties of the myocardium. This overcomes a key disadvantage in prior methods, wherein determining complex electrical muscle properties is typically both difficult and imprecise. Fourth, the calculations required to determine the volume of blood within the left ventricle [17] are computationally simple. Hence, the processor required to perform these calculations can be relatively simple. Fifth, there is no need to separate the admittance signal into a myocardial admittance component and a blood admittance component, as the total admittance signal, as shown in FIG. 8, is used to perform the calculations.

Using the aforementioned equation to determine the volume based on the admittance values, the SV is used to empirically normalize the mapping of admittance to the radius of the model cylinder of blood. Modeling the blood and myocardium structure of the left ventricle [17] is typically not entirely accurate, as the geometry of the ventricle [17] is likely not perfectly cylindrical. In order to model non-cylindrical geometries, a unit-less scale factor that maps the ideal cylindrical volume onto a non-cylindrical ventricular volume (β) is used. Incorporating β, the true stroke volume can be written as:

SV=β(V_{ED}−V_{ES}).

Since the volume of a cylinder can be expressed as a function of the admittance and the value of the admittance at the end of ventricular systole and the end of ventricular diastole are known, both the ESV and EDV can be calculated. Hence, both a calculated value for the stroke volume and well as a measured value for the stroke volume can be determined as previously described.

Using the unit-less β scale factor, the volume of the left ventricle [17] can be determined accurately for the entire cardiac cycle. Referring to FIG. 8, the volume within the left ventricle [17] can be calculated for each of the values on the admittance curve [46]. Hence, the volume can then be mapped to the blood pressure readings [44] for the entire cardiac cycle using the total admittance value.

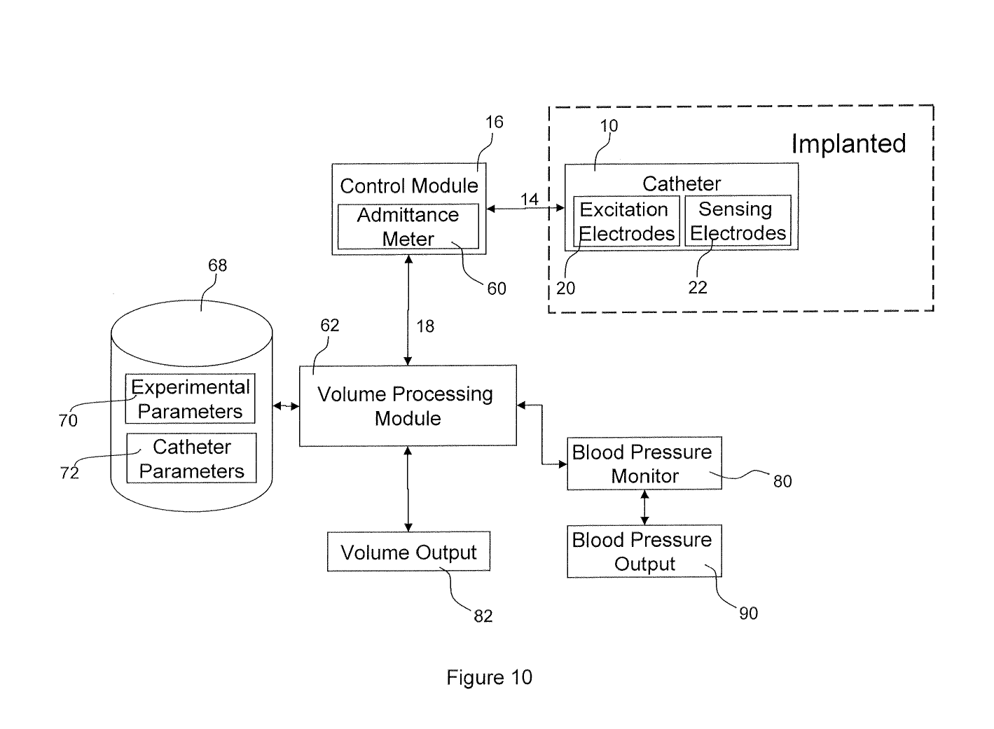

Referring to FIG. 10, a system for measuring the absolute volume within a physical lumen is illustrated. A control module [16] drives the excitation of the catheter [10], which is inserted into the lumen that is to be studied. For example, the catheter [10] is inserted through the aorta [11] into the left ventricle [17] and connected to the control module [16] via connecting line [14]. The control module [16] drives the excitation electrodes [20] of the catheter [10] and an admittance meter [60], or alternatively, a conductivity meter receives the electrical signal from the sensing electrodes [22]. The control module [16] can then communicate with a volume processing module [62] via a connection through port [18]. The volume processing module [62] may comprise memory and a processor and performs calculations based on the admittance signal received from the admittance meter [60]. The volume processing module [62] may take data from a database [68] of parameters associated with the catheter and electrodes being used [72], experimental parameters [70] such as the electrical properties of blood and myocardium and data from the blood pressure monitor [80] to perform the calculations as described above to determine the absolute volume of blood in the left ventricle [17].

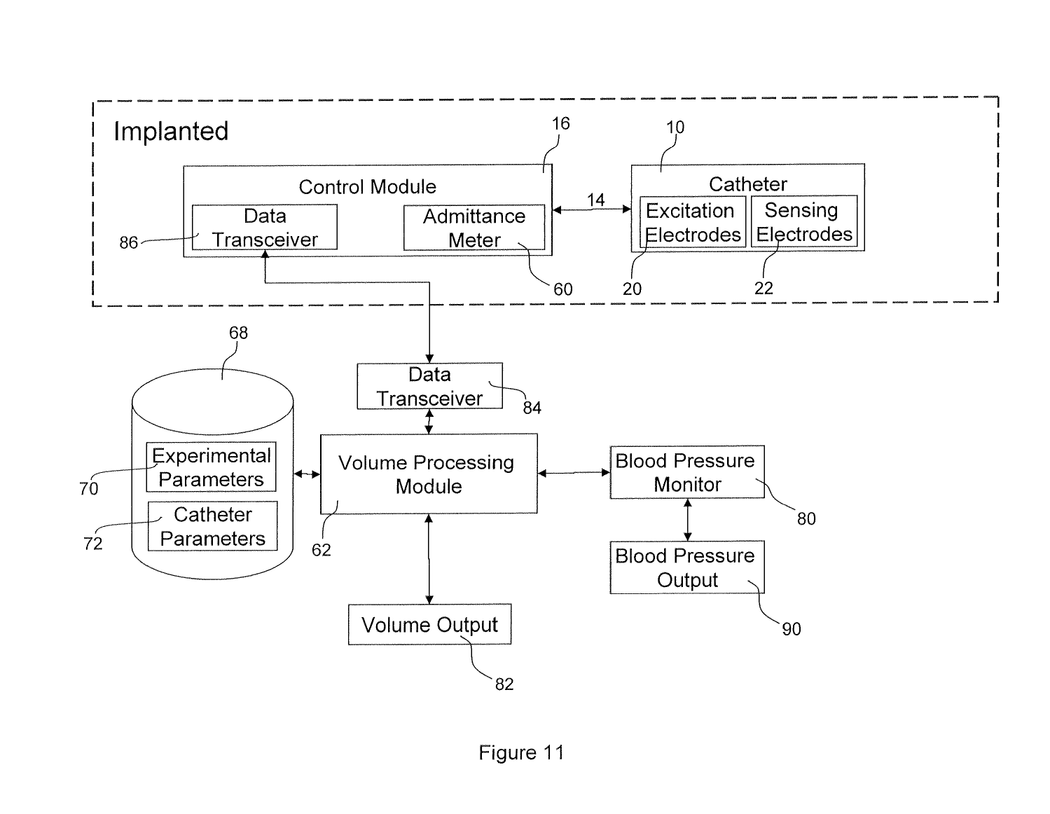

FIG. 11 shows a system similar to FIG. 10 wherein the connection between the control module [16] and the volume processing module [62] is achieved via a wireless connection. The control module [16] is further provided with a wireless transceiver [86] that makes a connection with a wireless transceiver [84]. An advantage of using a wireless connection between the control module [16] and the volume processing module [62] is that the use of cumbersome electrical connections can be avoided. A further advantage of using a wireless connection between the control module [16] and the volume processing module [62] is that the control module [16] can be implanted in the mammal being studied or fixed to the skin of the mammal in the location where the connecting line [14] enters the body. In this case, the catheter [10] and control module [16] are both fixed to the subject and the volume processing module [62], database [68] and blood pressure module [80] are located remotely. Alternatively, the blood pressure module [80] may be implanted or fixed on the subject. The blood pressure module may comprise a blood pressure sensor that can also be located directly on the catheter [10].

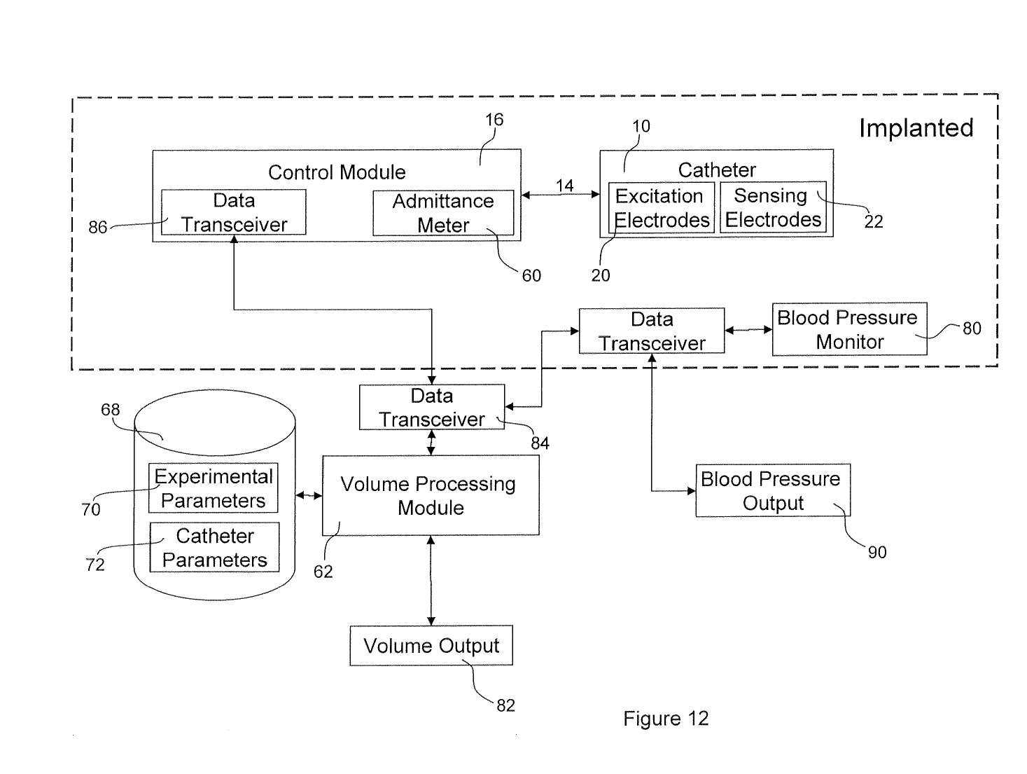

As an alternative to processing the admittance data to determine the absolute volume within the physical lumen in real time is to store the admittance data and pressure data in memory present in the implanted or externally fixed control module [16] or blood pressure module [80] for future calculation. An example diagram of such a system is shown in FIG. 12. The admittance data and pressure data may be stored together or may be stored separated separately with reference to time and compared later. For example, if the admittance data is measured using an implantable device and the blood pressure data is also determined using an implantable device; these signals may be stored and transmitted to the volume processing module [62] at a later time. By way of example, the implantable device may store several days or months of data. When the subject with the implant visits a physician, the physician may transfer the data to the volume processing module [62], where the data is analyzed to determine the subject's cardiac performance.

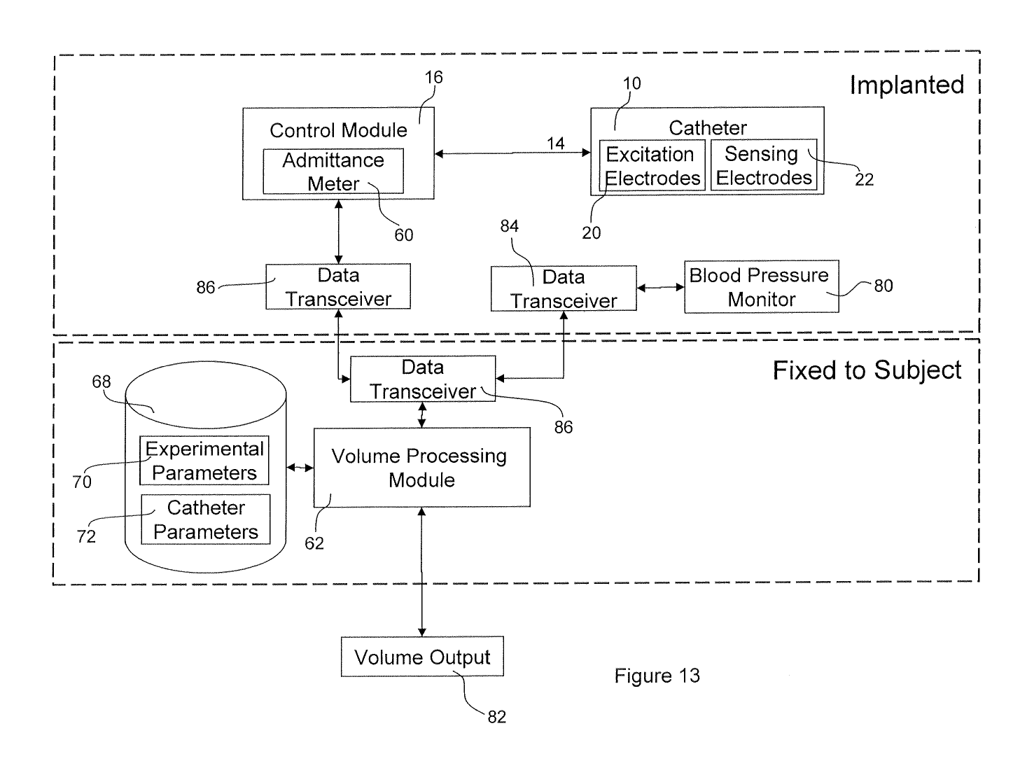

FIG. 13 shows an example diagram of a system where the volume processing module [62] is fixed to the subject being studied whereas the control module [16] is implanted and in communication with the catheter [10] via control line [14]. The volume processing module [62] may be connected either wirelessly or with a wired connection.

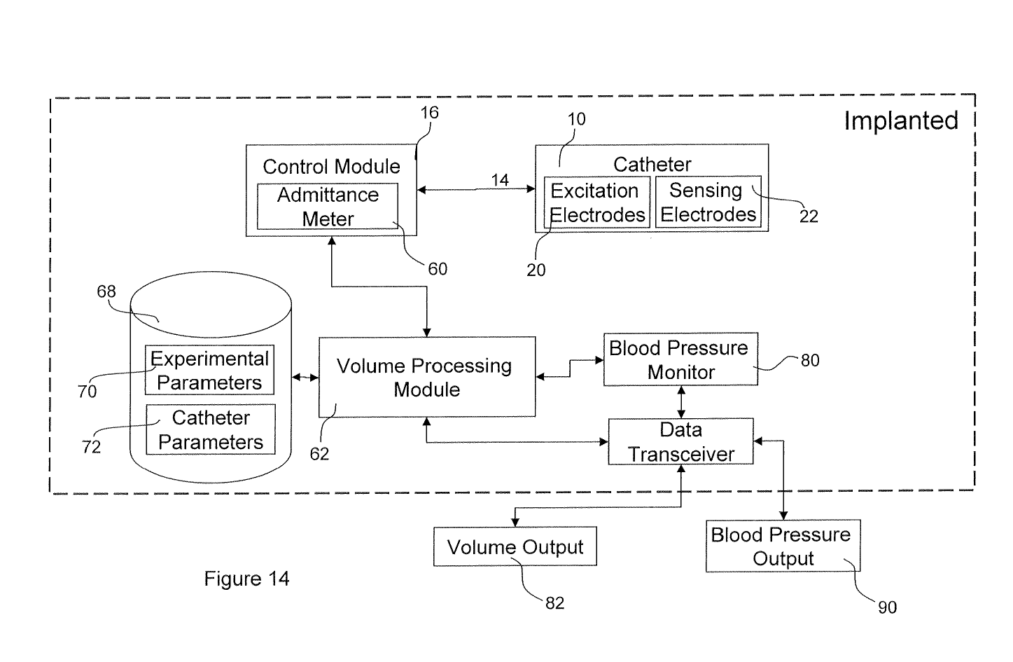

FIG. 14 shows an example diagram of a system where the volume processing module [62] is implanted in the subject, as is the control module [16], catheter [10] and blood pressure module [80]. Similarly to the above examples, the volume processing module [62] performs calculations based on the blood pressure monitor [80] and parameters within database [68]. The results of the volume calculations are transmitted either wirelessly or through a wired connection to a volume output [82]. The blood pressure may also be transmitted to a blood pressure output [90]. The transmission of data may be continuous or the volume processing module [62] and the blood pressure module [80] may store data and establish a connection to the volume output [82] and blood pressure output [90] upon request. Further, the volume processing module [62] and blood pressure module [80] may receive instructions that could include, for example, the sampling rate, when to begin or end sampling, updating parameters within database [68], blood pressure monitor parameters [80], and firmware updates for the implanted modules. Similarly, the volume processing module [62] may provide status updates such as, by way of example, battery power, and warnings regarding inaccurate readings or a lost connection, abrupt changes in blood pressure or admittance.

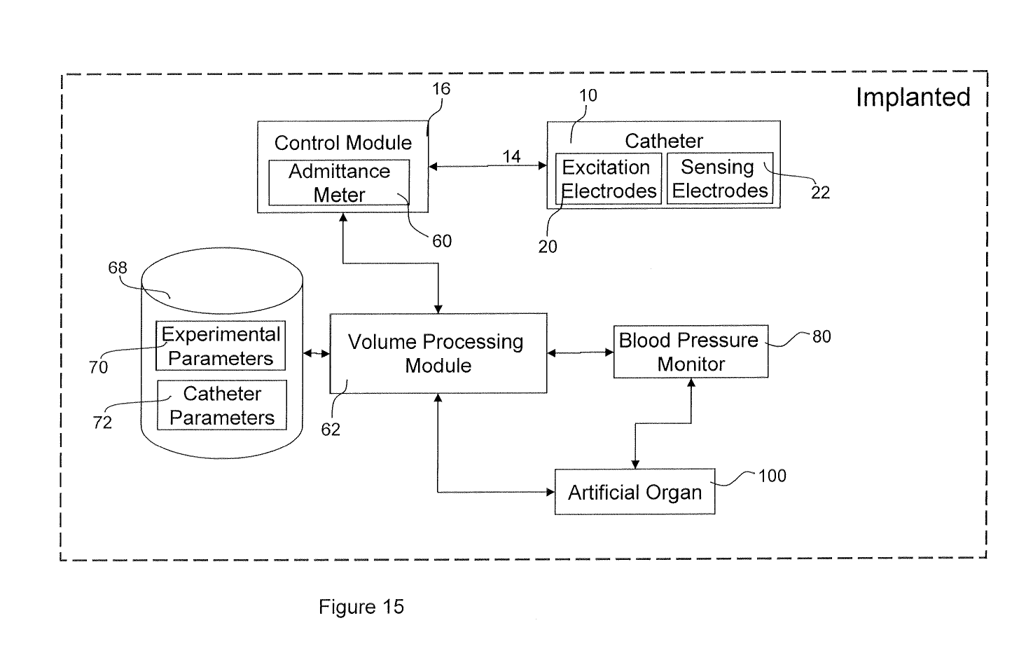

FIG. 15 shows the implanted system of FIG. 14 wherein an artificial organ is also implanted in the subject. The volume calculations yielded from the volume processing module [62] and the blood pressure module [80] could be transmitted to the artificial organ via a wired or wireless connection. The artificial organ could then adjust its function based on the volume calculations and blood pressure measurements. For example, in the case of an implanted artificial bladder, the system could determine, via a catheter implanted in the bladder, the extent to which the bladder is full. For example, when the bladder reaches a certain threshold of its maximum capacity, it can indicate to the subject that the bladder should be emptied. Alternatively, a catheter [10] implanted in the left ventricle [17] of the heart could be used to monitor the volume of blood being pumped in the heart to determine at what rate insulin is released in a controlled release system.

It will be appreciated that any application or module exemplified herein may include or otherwise have access to computer readable media such as storage media, computer storage media, or data storage devices (removable and/or non-removable) such as, for example, magnetic disks, optical disks, or tape. Computer storage media may include volatile and non-volatile, removable and non-removable media implemented in any method or technology for storage of information, such as computer readable instructions, data structures, program modules, or other data, except transitory propagating signals per se. Examples of computer storage media include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by an application, module, or both. Any such computer storage media may be part of the volume processing module [62], control module [16], blood pressure module [80] or any other device, accessible or connectable thereto. Any application or module herein described may be implemented using computer readable/executable instructions that may be stored or otherwise held by such computer readable media.

Although the above has been described with reference to certain specific example embodiments, various modifications thereof will be apparent to those skilled in the art without departing from the scope of the claims appended hereto.

1. A method for determining absolute volume of a physical lumen, the method comprising:
obtaining a plurality of admittance measurements;
determining a first admittance value representative of the lumen at a peak volume;
determining a second admittance value representative of the lumen at a lowest volume; and
determining an instantaneous volume of the lumen using the first and second admittance values and at least one predetermined calibration parameter.

2. The method of claim 1 wherein the plurality of admittance measurements are obtained using a catheter.

3. The method of claim 2 wherein the catheter has more than 4 electrodes.

4. The method of claim 1 wherein the plurality of admittance measurements are transmitted to a volume processing module to determine the instantaneous volume.

5. The method of claim 1 wherein the plurality of admittance measurements are obtained using an implantable device.

6. The method of claim 5 wherein the implantable device wirelessly transmits the plurality of admittance measurements to an externally located volume processing module.

7. The method of claim 1 wherein the volume being measured is the volume of blood in a left ventricle.

8. The method of claim 1 wherein only a conductance component of the admittance is measured.

9. The method of claim 7 wherein admittance of an end diastolic volume (EDV) and admittance of an end systolic volume (ESV) are determined by comparing a blood pressure plot to an admittance curve over at least a portion of a cardiac cycle.

9. (canceled)

10. The method of claim 1 wherein the physical lumen is not a native physical lumen.

11. The method of claim 10 wherein the physical lumen is an artificial lumen.

12. The method of claim 11 wherein the artificial lumen is an artificial heart.

13. A computer readable medium for determining absolute volume of a physical lumen, the computer readable medium comprising computer executable instructions for:
obtaining a plurality of admittance measurements;
determining a first admittance value representative of the lumen at a peak volume;
determining a second admittance value representative of the lumen at a lowest volume; and
determining an instantaneous volume of the lumen using the first and second admittance values and at least one predetermined calibration parameter.

14. A system comprising a processor coupled to a memory, the memory comprising computer executable instructions for:
obtaining a plurality of admittance measurements;
determining a first admittance value representative of the lumen at a peak volume;
determining a second admittance value representative of the lumen at a lowest volume; and
determining an instantaneous volume of the lumen using the first and second admittance values and at least one predetermined calibration parameter.

15. The system of claim 14 further comprising a catheter configured to provide the plurality of admittance measurements to the processor.

16. The system of claim 15 wherein the catheter has more than 4 electrodes.

17. The system of claim 14 wherein the plurality of admittance measurements are obtained using an implantable device.

18. The system of claim 17 wherein the implantable device wirelessly transmits the plurality of admittance measurements to the processor and wherein the processor is located external to the implantable device.

19. The system of claim 14 wherein pressure signals and the plurality of admittance measurements are collected using an implanted device and stored before being transmitted to the processor.

20. The method of claim 1 wherein pressure signals and the plurality of admittance measurements are collected using an implanted device and stored before being transmitted to a volume processing module.
